(19) United States
(12) Patent Application Publication
(10) Pub. No.: US 2005/0175595 A1
(43) Pub. Date: Aug. 11, 2005

(54) Method for reinstalling control system for antigenic homeostasis of mammalian organisms (effect of Kukharchuk-Radchenko-Sirman)

(76) Inventors: Oleksandr L. Kukharchuk, Chernovsty (UA); Viktor V. Radchenko, Kiev (UA); Viktor M. Sirman, Kiev (UA)

Correspondence Address: DARBY & DARBY P.C., P. O. BOX 5257, NEW YORK, NY, 10150-5257, US

(21) Appl. No.: 11/073,498

(22) Filed: Mar. 3, 2005

Related U.S. Application Data
(63) Continuation of application No. PCT/UA03/00030, filed on Sep. 1, 2003.

(30) Foreign Application Priority Data
Sep. 3, 2002 (UA) 2002097178

(51) Int. Cl. A61K 45/00

(52) U.S. Cl. 424/93.7

(57) ABSTRACT

The invention relates to a novel method for induction of immunological tolerance in a mammal with embryonic pluripotent progenitor cells (EPPC) via the formation of a new basis of immunocompetent cells with simultaneous de novo installation of the control system for the antigenic homeostasis (Kukharchuk-Radchenko-Sirman effect). This method can be used for allo- and xenotransplantation of organs and tissues and for treatment of various diseases.

### CROSS-REFERENCE TO RELATED APPLICATIONS

This application is a continuation of International Patent Application No. PCT/UA2003/00030 filed Sep. 1, 2003 (published in Russian on Mar. 18, 2004 as WO 2004/022079), and claims the benefit of Ukrainian Patent Application UA No. 2002097178 filed Sep. 3, 2002, both of which are incorporated herein by reference in their entirety.

### FIELD OF THE INVENTION

The present invention relates to a novel method for induction of immunological tolerance in a mammal with embryonic pluripotent progenitor cells (EPPC) via the formation of a new basis of immunocompetent cells with simultaneous de novo installation of the control system for the antigenic homeostasis (Kukharchuk-Radchenko-Sirman effect). This method can be used for allo- and xenotransplantation of organs and tissues and for treatment of various diseases.

### BACKGROUND OF THE INVENTION

It is currently believed that, during transplantation in a mammal, an allograft rejection occurs as a result of expression of antigens, which are not compatible with the major histocompatibility complex (MHC, HLA) of a recipient. In addition, the unity of the immune response in allotransplantation is determined by the fact that foreign MHC (HLA) molecules directly activate T-cells of a recipient, i.e., a molecular basis of the allotransplant rejection reaction is the interaction of receptors of T-lymphocytes with MHC (HLA) molecules, predominantly HLA-DR (Roitt A., Brostoff J, Meil D. Immunology/Translation from English by V. I. Kandoror, A. N. Mats, L. A. Pevnitskyy, M. A. Serova.-M.: Mir, 2000.-p. 592.). Current methods used to prevent transplant rejection include non-specific and specific immune suppression, a non-reactivity induction to a transplant by means of a directed influence on the cytokine regulation of the immune response, modeling of the ratio between Th1 and Th2 type lymphocytes, application of antigens to CD3⁺-cells, etc. (see, e.g., WO 9930730 A 1. Tremblay, Jacques, P. Methods and Compositions for Cell Transplantation to the Host.-Inventions of Countries of the World.-2000.-Vol. 8, No. 12.-P. 70; U.S. Pat. No. 5,914,314 A. Falk, Rudolf Edgar, Asculai, Samuel S. Form of Hyaluronic Acid and Medical Agent Applied to Decrease the Rejection Reaction of Transplanted Organs in Mammals.-Inventions of Countries of the World.-2000.-Vol. 8, No. 12.-P. 50; U.S. Pat. No. 5,916,559 A. Strom, Terry B. Agents Application Specific for Interleukin-2 Receptor for Transplant Rejection Treatment.-Inventions of Countries of the World-2000.-Vol. 8, No. 12.-P. 54; 6 A 61 K 39/385. Karpov I. A., Kopyltsev V. N., Ckaletskyy N. N. Method of Transplantation of Organs and Tissues.-1998.-RU BI No. 32.-P. 340; WO 6A 61K 31/70. Simon, Paul M.; Mequire, Edward, J. Methods and Oligosaccharides to Weaken the Transplant Rejection.-Inventions of Countries of the World.-2000.-Vol. 8, No. 1.-P. 71).

During last years a great variety of new reports about embryonic stem cell (ESC) biology and application prospects of the latter in practical medicine have been published in scientific literature. The main approach used in many of these reports is a transformation of ESC being at the stages of toti-, pluri- or multipotancy into specialized cells in the zone of tissue damage in different organs (see, e.g., Civin C. I. Human Pluripotent Stem Cells: Science Fiction Poses no Immediate Dangers//Stem Cells.-2000.-Vol. 18.-P. 4-5.; Takatsugu Y., Masahide Y., Seiji K., Yoko K., Yoshiyuki N., Shigeaki I., Yukio T. In Vitro Differentiation of Embryonic Stem Cells into Hepatocyte-like Cells Identified by Cellular Uptake of Indocyanine Green II Stem Cells.-2002.-Vol. 20.-P. 146-154.).

It is believed that totipotent embryonic stem cells (ESC) and embryonic pluripotent progenitor cells (EPPC), under the influence of certain growth factors and cytokines are able to differentiate into the cells of any tissue, including the cells of the immune and blood-forming system (Schuldiner M., Yanuka O., Itskovitz-Elder J. Effects of 8 Grown Factors on the Differentiation of Human Embryonic Stem Cells II Proc. Nat. Acad. Sci. USA.-2000.-Vol. 97.-P. 111307-11312.). The property of a structural and functional composition of the latter is that these systems represent a dynamic population of progenitor cells fixed on stromal elements, and differentiated effector cells, which perform their functions beyond specific organs. In particular, such property of a structural and functional organization of the blood system leads to the so called “blood chimerism”, when, in the organism of an immunologically exhausted recipient, after the bone marrow transplantation, erythrocytes with antigens of different blood groups co-exist. The blood chimerism may last for 90-420 days, and the dynamics of blood groups change is characterized by a gradual decrease of the number of the donor's erythrocytes with a permanent increase of the number of erythrocytes, which have group antigens of the recipient (Zotikov E. A. Karl Landshteyer and his Heritage//Hematology and Transphysiology-2001.-V. 46, No. 5.-P. 25-27.) that is a profound demonstration of the law of conservation of the cell mass, stromal syngenic preference and allogenic inhibition (Vershygora A. E. General Immunology.-K.: Higher School Publishers, 1989.-P. 736; Shevchenko Y. L., Zhyburt E. B. Safe Blood Transfusion.-SPb, Peter, 2002.-320 p.; Kikuya S., Hirroko H., Junji I., Yasushi A., Shigeru T., Shinryu L., Takashi N., Susumu I. Major Histocompatibility Complex Restriction between Hematopoietic Stem cells and Stromal Cells in vitro//Stem Cells.-2001.-Vol. 19.-P. 46-58.).

It is well recognized that an organ's stroma is not only its spatial framework, but represents a population of biologically active elements, which provide for all aspects of parenchyma cells functioning. In human and mammalian embryogenesis, the formation of about 300 types of specialized cells occurs owing to the cellular pool of embryonic petals (ecto-, ento-, and mesoderma), and mesenchyme with multiple metabolic, signaling, mechanical and morphological functions. Clonogenic proliferation of stem and progenitor mesenchymal cells, their migration and synthesis of biologically active substances regulate organogenesis; provide for an advanced development of blood and lymphatic vessels and stroma formation for future organs. During the embryogenesis mesenchymal cells produce growth factors (HGF, TGF-α, EGF, KGF), for which, on membranes of parenchymatous progenitor cells, receptors are expressed, and, in a differentiated adult tissue, the stromal net generates signals to support vital activity and proliferation of regional stem cells (SCF, HGF, IL-6, IL-7, IL-8, IL-11, IL-12, IL-14, IL-15, M-CSF, LIF etc.). More than 20% of mesenchymal stem and progenitor cells, after administration into the blood of an adult human, are captured by the stroma of the blood-forming tissue and parenchymatous organs, that is currently used for increase of efficiency of bone marrow recolonization in oncological patients after irradiation, when immature mesenchymal cells are administrated into the blood along with hemopoietic stem/progenitor cells (Dennis J. E., Charbord P. Origin and Differentiation of Human and Murine Stroma II Stem Cells.-2002.-Vol. 20.-P. 205-214.; Huss R. Isolation of Primary and Immortalized CD34-Hematopoietic and Mesenchymal Stem Cells from Various Sources II Stem Cells.-2000.-Vol. 18.-P. 1-9.; Taketoshi K., Muneo I., Hiroko H., Naoya I., Takashi E., Ryokei O., Hirokazu I., Susumu I. Crucial Role of Donor-Derived Stromal Cells in Successful Treatment for Intractable Autoimmune Diseases in MRL/lpr Mice by BMT via Portal Vein II Stem Cells.-2001.-Vol. 19.-P. 226-235.; Tian-Xue F., Hiroko H., Tie-Nan J., Cheng-Ze Y., Zhe-Xiong L., Shu-Bin G., Yun-Ze C., Biao F., Guo-Xiang Y., Qing L., Susumu I. Successful Allogeneic Bone Marrow Transplantation (BMT) by Injection of Bone Marrow Cells via Portal Vein: Stromal Cells as BMT-Facilitating Cells//Stem Cells.-2001.-Vol. 19.-P. 144-150.).

It has been demonstrated that ESC and progenitor cells at pluripotance level are able to differentiate into specialized cells of a type or form that depend on the microenvironment (Fandrich F., Lin X., Chai G. X. Preimplantation-Stage Stem Cells Induce Long-Term Allogeneic Graft Acceptance Without Supplementary Host Conditioning//Nat. Med.-2002.-Vol. 8.-P. 171-178.; Hawley R. G., Sobieski D. A. New Feature: Stem Cells in the News//Stem Cells.-2002.-Vol. 20.-P. 103-104; Stem Cells in the News: Robert G. Hawley, Donna A. Sobiesky, 2002). However, prior to the present invention, it has not been foreseen or suggested to use embryonic stem cells (ESC) or embryonic pluripotent progenitor cells (EPPC) for replacement in adult mammals of the part of the immune system that controls antigenic homeostasis.

### SUMMARY OF THE INVENTION

The present invention provides a novel method for induction of immunological tolerance in a mammal with embryonic pluripotent progenitor cells (EPPC) via the formation of a new basis of immunocompetent cells with simultaneous de novo installation of the control system for the antigenic homeostasis (Kukharchuk-Radchenko-Sirman effect).

The present invention also provides a method of reinstallation of the control system for antigenic homeostasis of a mammalian organism, characterized in that, instead of the antigen selection of donor tissues and immune suppression, the immunological tolerance of the recipient to allo- or xenotransplants is achieved by the administration of embryonic pluripotent progenitor cells (EPPC) which form a new basis of immunocompetent cells controlling the antigenic homeostasis of the organism.

In a specific embodiment, according to the methods of the present invention, the EPPC are administered in megadoses. In a preferred embodiment, the megadoses of EPPC correspond to doses of at least one million (10⁶) (and more preferably several million) of viable nucleated cells per 1 ml of the cell suspension.

In a specific embodiment, EPPC are administrated intravenously or intraperitoneally.

The present invention also provides the use of the methods of the invention for allo- or xenotransplantation of an organ or tissue in a mammal.

In a preferred embodiment, when used for allo- or xenotransplantation of an organ or tissue in a mammal, EPPC are administered shortly prior to transplantation.

The present invention also provides the use of the methods of the invention for the treatment of various diseases, including, without limitation, autoimmune diseases (glomerulonephritis, rheumatoid arthritis, chronic active hepatitis, etc); neurodegenerative diseases, wherein an autoimmune process is the main pathogenic component (e.g., Alzheimer's disease, multiple sclerosis, amyotrophic lateral sclerosis, etc.); immune infertility; myocardial infarction; stroke; Parkinson's disease; hypo- and aplastic anemia; immune miscarriages; endocrine diseases associated with hypofunction of inner secretion glands, including diabetes mellitus; various forms of osteoporosis; female and male climax; and radiation sickness.

### BRIEF DESCRIPTION OF THE DRAWINGS

FIGS. 1-8 show the effect of administration of embryonic pluripotent progenitor cells (EPPC) on spleen allotransplant engraftment in rats.

FIG. 1 is a photograph of an animal that has not received EPPC. One month after the operation under Nembutal narcosis a midline laparotomy was performed. Epiploon was turned out into operative wound, and allospleen was closed into it. The allospleen is encapsulated by epiploon.

FIG. 2 is a photograph of an animal that has received EPPC. One month after the operation under Nembutal narcosis a midline laparotomy was performed. The allospleen is located in the thickness of the non-inflamed epiploon. Blood vessels are connected to the allospleen.

FIG. 3 is a photograph showing the results of opening of the inflamed epiploon recess in the control rat one month after spleen allotransplantation. Inside of the detritic mass a pale-brown allospleen is located that freely lies on the connective tissue capsule and is not vascularized by the epiploon vessels.

FIG. 4 is a photograph showing the results of an attempt to remove the allospleen from the epiploon of the experimental rat. Hemorrhage signs are visible around the affected epiploon. The allospleen has a normal color, a tight contact with the epiploon as a result of vascularization by the vessels.

FIG. 5 is a photograph of a control animal that has not received EPPC. Two months after the operation under Nembutal narcosis a midline laparotomy was performed. Epiploon was turned out into the operation wound; the allospleen was closed to the epiploon. The allospleen is located in the recess that is created by the inflamed epiploon.

FIG. 6 is a photograph of an animal that has received EPPC. Two months after the operation, a midline laparotomy was performed. The allospleen is located in the thickness of a normal epiploon and vascularized.

FIG. 7 is a photograph showing the results of opening of the inflamed epiploon recess in the control rat two months after spleen allotransplantation. Inside the recess there are only detritic masses, and no allospleen. Walls of the inflamed recess are formed by the rough connective tissue.

FIG. 8 is a photograph of the allospleen of the animal which received EPPC. Two months after the operation. The spleen is vascularized and has a normal color. There are no inflamed changes of the epiploon.

FIG. 9 shows general arrangement of the experiment for the induction of immunological tolerance to spleen allotransplants by EPPC:

A—EPPC were isolated from the embryos received from a female rat and intravenously administered to the recipient rat. The control animal received a necessary doze of Hanks solution. Pair allotransplantation was performed: a half of the control rat spleen was transplanted into the epiploon of the EPPC recipient rat, and another part of the spleen of the latter—into the epiploon of the control animal.

B—Two months after the operation, a midline laparotomy was performed, during which an allospleen engraftment in the EPPC rat and an allospleen rejection in the control rat were found. Step C of the experiment was performed immediately after this.

C—A half of the spleen of the control rat which remained in situ after the first operation (see stage A), was transplanted into the epiploon of the EPPC recipient animal, and the remaining spleen of the EPPC recipient rat was closed into the epiploon of the control animal. A piece of the recipient rat's skin was transplanted to the control animal and, vise versa, a piece of the control animal's skin was transplanted to the experimental rat that received EPPC.

D—Two weeks after allotransplantation of the second halves of the spleen in experimental animals that received EPPC, the skin allotransplant was rejected, but the allospleen showed engraftment, whereas, in the control group of rats, the rejection of skin and spleen allotransplants was observed.

FIGS. 10-15 show experimental results for the induction of immunological tolerance to spleen allotransplants by EPPC:

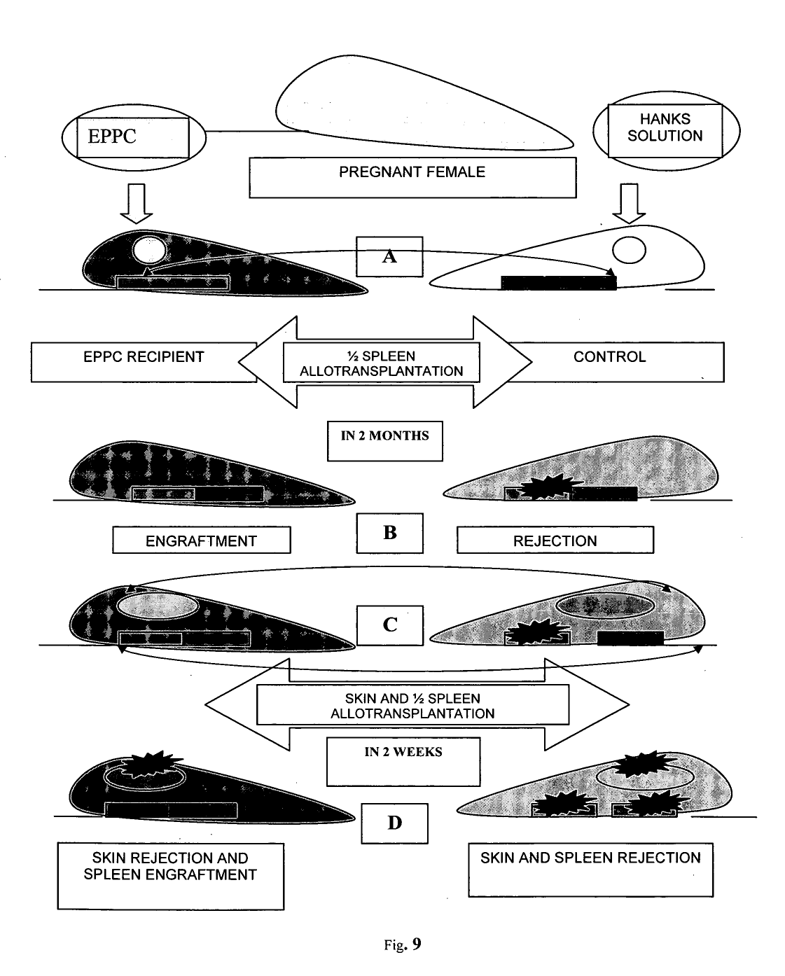

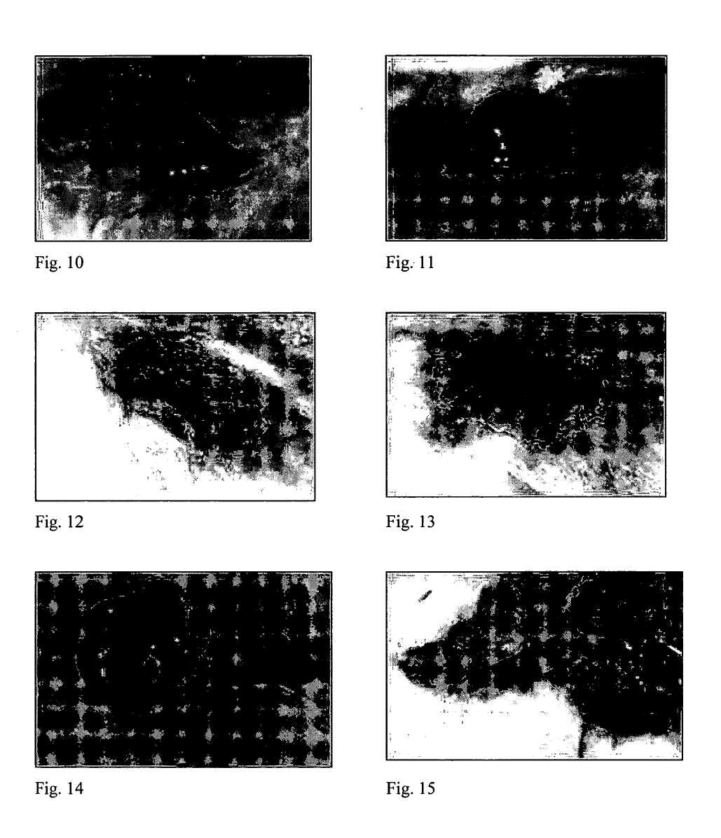

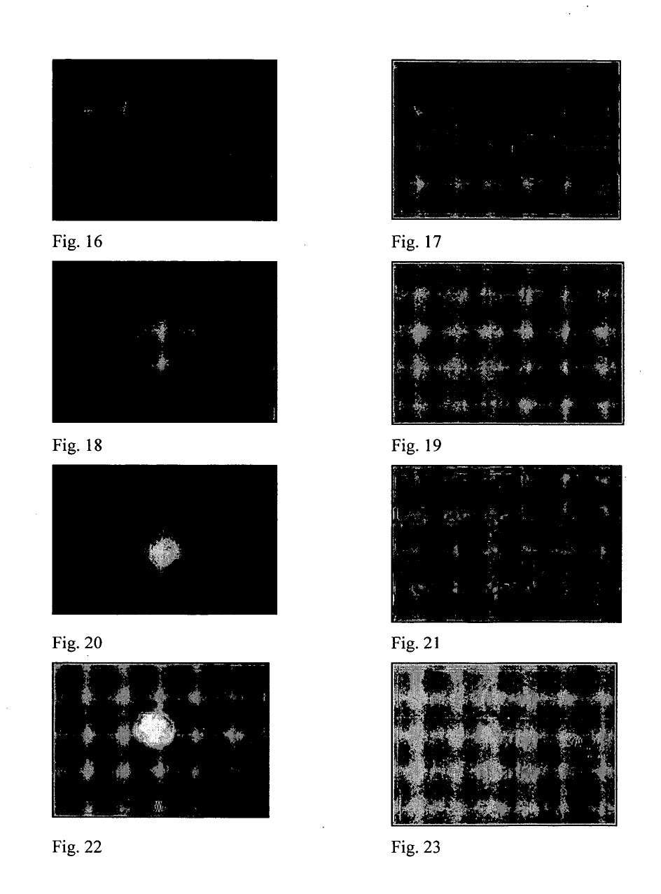

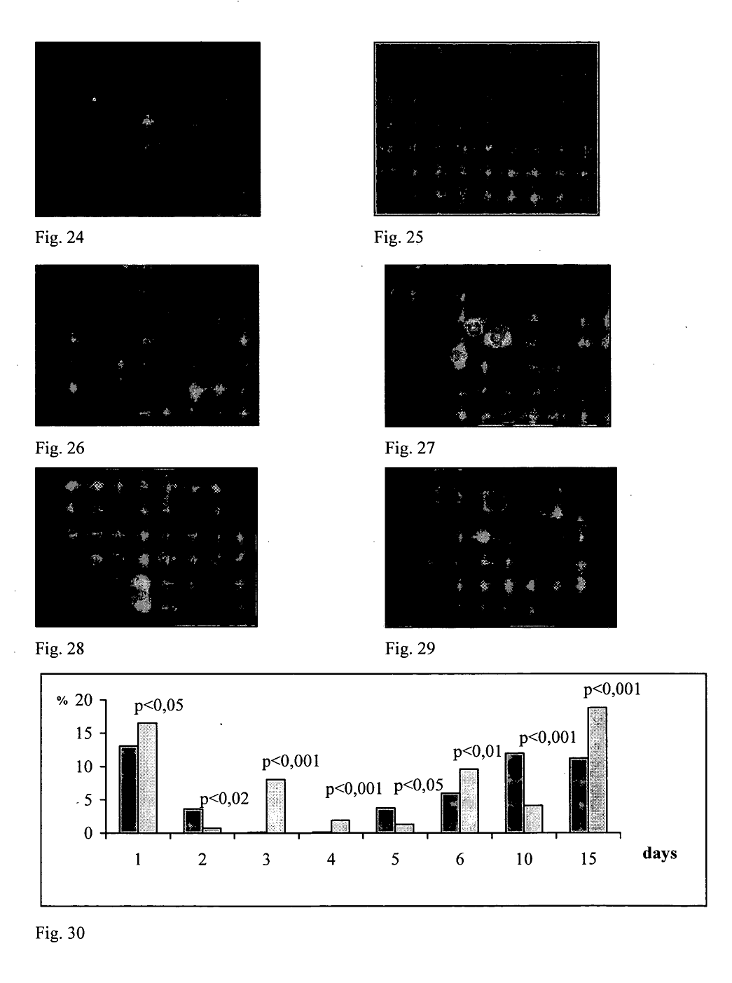

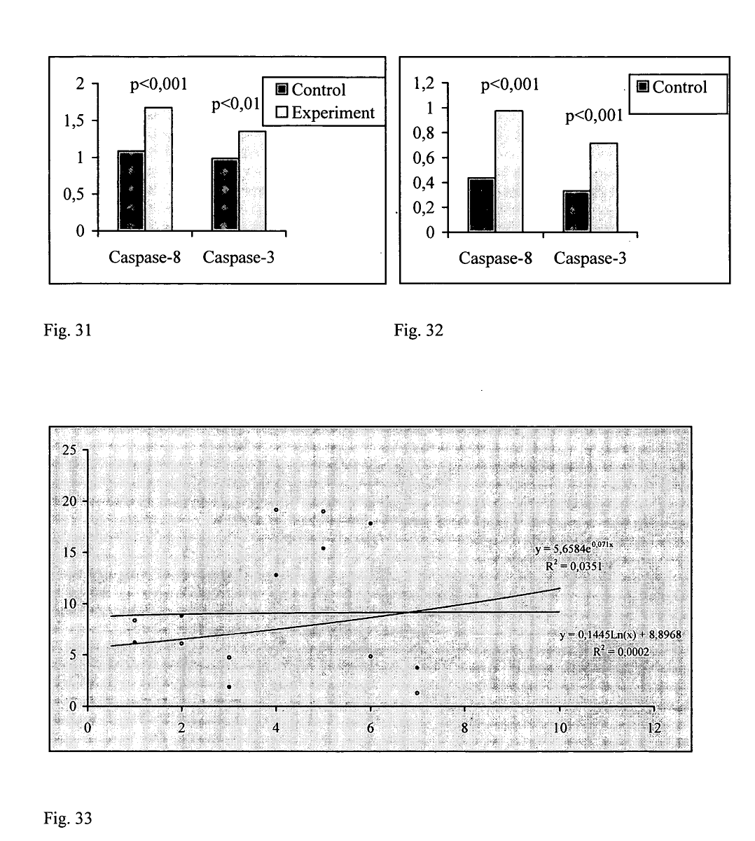

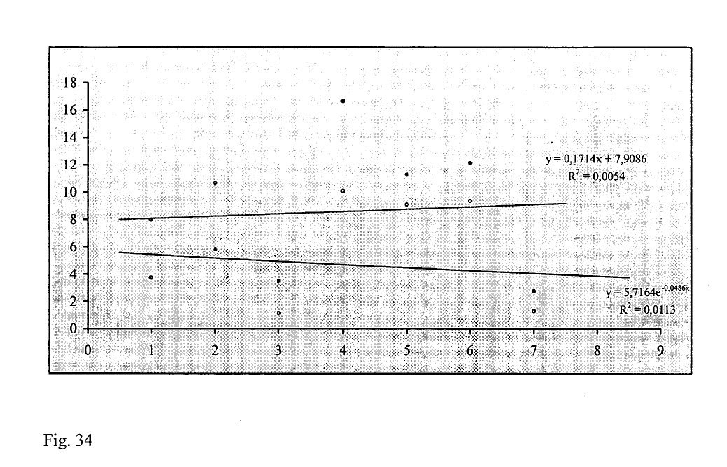

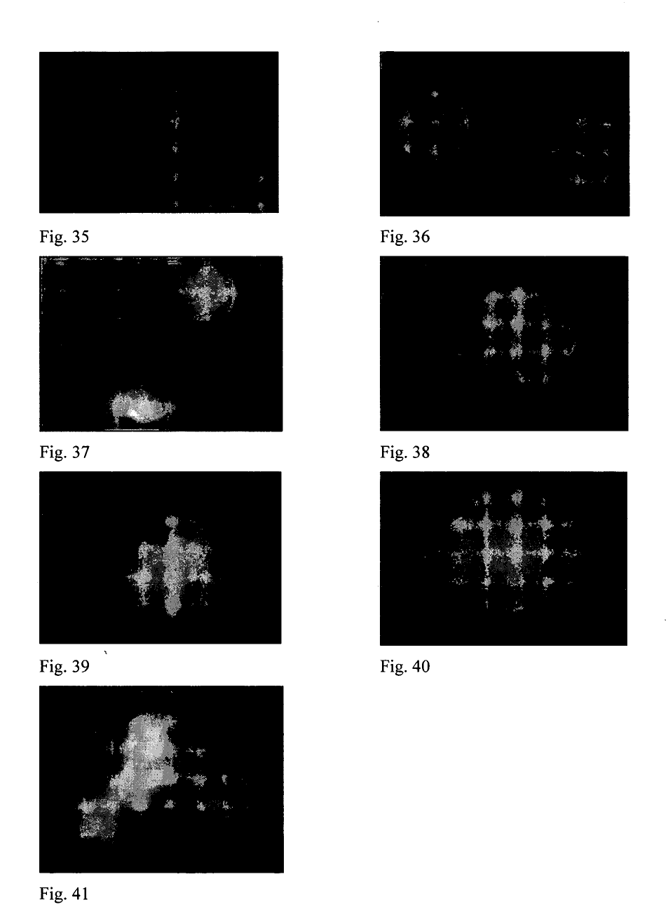

FIGS. 10-11 are photographs demonstrating stage B of the general arrangement shown in FIG. 9: in the rat that received EPPC, the allospleen engraftment occurs, while in the control group animal, it is rejected. (FIG. 10—EPPC administration, FIG. 11—control).

FIGS. 12-13 are photographs demonstrating stage D of the general arrangement shown in FIG. 9: skin allotransplants are rejected both in the control and experimental animals. (FIG. 12—EPPC administration, FIG. 13—control)

FIGS. 14-15 are photographs demonstrating stage D of the general arrangement shown in FIG. 9: repeated allotransplantation of the second half of allospleen of the rat that received EPPC results in its engraftment, whereas, in the control animal, an allospleen rejection occurs. (FIG. 14—EPPC administration, FIG. 15—control)

FIGS. 16-25 show PKH 67-positive cells in rat thymus:

FIG. 16 is a luminescent microscopy pattern of a basal suspension of EPPC before their administration into the recipient rats.

FIG. 17 is a phase-contrast microscopy pattern of a basal suspension of EPPC before their administration into recipient rats.

FIGS. 18, 20, 22, 24 are luminescent microscopy patterns that identify PKH 67-positive cells in thymus.

FIGS. 19, 21, 23, 25 are phase-contrast microscopy patterns of thymus cells.

FIGS. 26-32 show apoptosis and caspase activity in the immune system organs of rats with spleen allotransplantant, which received EPPC:

FIG. 26 shows apoptosis in thymus of rats, which received EPPC.

FIG. 27 shows apoptosis in bone marrow of rats, which received EPPC.

FIG. 28 shows apoptosis in spleen of rats, which received EPPC.

FIG. 29 shows apoptosis in lymph nodes of rats, which received EPPC.

FIG. 30 shows apoptosis dynamics (%) of thymus cells in the control animals (dark columns) and rats, which received EPPC (light columns).

FIG. 31 shows caspase-8 (a) and caspase-3 (b) activity in thymus of control animals (dark columns) and rats, which received EPPC (light columns) (measure unit−UE/1 mg of protein).

FIG. 32 shows caspase-8 (a) and caspase-3 (b) in bone marrow of control rats (dark columns) and rats, which received EPPC (light columns) (measure unit−UE/1 mg of protein).

FIGS. 33-34 show lines of intensity trend for apoptosis of thymus and bone marrow cells in the control animals and rats, which received EPPC during spleen allotransplantation:

FIG. 33 shows lines of a percentage trend for apoptotic cells in thymus of the control rats (dotted line) and animals, which received EPPC (solid line).

FIG. 34 shows lines of percentage trend for apoptotic cells in thymus of the control rat (dashed line) and animals, which received EPPC (solid line).

FIGS. 35-41 show apoptosis of EPPC in a mixed suspension (from two different embryos):

FIG. 35 is a suspension A. EPPC stained with PKH 67.

FIG. 36 is a suspension B. EPPC stained with propidium iodide.

FIG. 37 is a suspension A. EPPC stained with PKH 67 and propidium iodide.

FIG. 38 is an EPPC “A+B” mixed suspension. Classic apoptosis of the B cell.

FIG. 39 is an EPPC “A+B” mixed suspension. Cells A: chromatin condensation. Blebs.

FIG. 40 is an EPPC “A+B” mixed suspension. Apoptotic nuclear decay of the B cell.

FIG. 41 is an EPPC “A+B” mixed suspension after 18 hours of incubation: deposition of apoptotic cells and bodies.

### DETAILED DESCRIPTION OF THE INVENTION

The present invention is based on a discovery that the immunological tolerance to allogenic transplants in a mammal can be induced by administering megadoses of embryonic pluripotent progenitor cells (EPPC) (Kukharchuk-Radchenko-Sirman effect). In contrast to previously known methods for prevention of transplant rejection reaction, in the present invention, the immunological tolerance is achieved by means of formation of a new basis of immunocompetent cells of the control system for antigen homeostasis in the organism.

The present invention provides a novel method for induction of immunological tolerance in a mammal with embryonic pluripotent progenitor cells (EPPC) via the formation of a new basis of immunocompetent cells with simultaneous de novo installation of the control system for the antigenic homeostasis (Kukharchuk-Radchenko-Sirman effect).

The present invention also provides a method of reinstallation of the control system for antigenic homeostasis of a mammalian organism, characterized in that, instead of the antigen selection of donor tissues and immune suppression, the immunological tolerance of the recipient to allo- or xenotransplants is achieved by the administration of embryonic pluripotent progenitor cells (EPPC) which form a new basis of immunocompetent cells controlling the antigenic homeostasis of the organism.

In a specific embodiment, according to the methods of the present invention, the EPPC are administered in megadoses. In a preferred embodiment, the megadoses of EPPC correspond to doses of at least one million (10⁶) (and more preferably several million) of viable nucleated cells per 1 ml of the cell suspension.

In a specific embodiment, EPPC are administrated intravenously or intraperitoneally.

The present invention also provides the use of the methods of the invention for allo- or xenotransplantation of an organ or tissue in a mammal.

In a preferred embodiment, when used for allo- or xenotransplantation of an organ or tissue in a mammal, EPPC are administered shortly prior to transplantation.

The present invention also provides the use of the methods of the invention for the treatment of various diseases, including, without limitation, autoimmune diseases (glomerulonephritis, rheumatoid arthritis, chronic active hepatitis, etc); neurodegenerative diseases, wherein an autoimmune process is the main pathogenic component (e.g., Alzheimer's disease, multiple sclerosis, amyotrophic lateral sclerosis, etc.); immune infertility; myocardial infarction; stroke; Parkinson's disease; hypo- and aplastic anemia; immune miscarriages; endocrine diseases associated with hypofunction of inner secretion glands, including diabetes mellitus; various forms of osteoporosis; female and male climax; and radiation sickness.

The present invention is further described by way of the following particular example. However, the use of such example is illustrative only and is not intended to limit the scope or meaning of the present invention or of any exemplified term.

### EXAMPLE 1

### The Use of EPPC Megadoses to Induce Immunological Tolerance to Skin and Spleen Allografts in Sexually Mature Rats

### Methods

In order to isolate EPPC, pregnant female rats (certificate of the nursery of A.A. Bogomolets Institute of Physiology of NAS of Ukraine) were anesthetized (pentobarbital sodium−40 mg per 1 kg of body weight) at developmental stages 11-13 of an embryo according to Astaurov (Objects of Development Biology/Edited by B. L. Astaurov.-M.: Science, 1975.-580 p.). After an aseptic preparation of the operative field (96% ethyl alcohol, iodine), the midline laparotomy was performed along linea alba. Both uterine horns were placed in the operative wound and cut with sterile scissors across (close to the embryos). The latter were placed on a sterile Petri dish with a cooled up to 4° C. Hanks' solution with gentarnicin (final gentamicin concentration−0,001%). After a triple rinse, EPPC were isolated from embryos according to the inventors' original methodology (UA Patent Application No. 20022097445). EPPC suspension was filtered through a capron filter (diameter of pores is 200 μm). An equal volume of 5% dimexide solution pre-filtered through a cellular filter with pores having diameter of 0.20 μm was added to the filtrate.

After the skin transplantation, the experimental rats were intravenously injected with EPPC suspension (viability control was performed using a light microscope with a trypan blue cells staining) in a megadose (several million of viable nucleated cells in 1 ml of the cell suspension). The EPPC output is 80×10⁹/l. Calculations prove that, e.g., a maximal dose of hematopoietic cells of human embryonic liver, in case of intravenous administration of 4.0 ml suspension containing 2.5×10⁸ cells per ml, results in 0.2 cells per μl concentration of the latter in the patient's blood (without taking into account the tissue pool). The dose used in the present experiments (80×10⁹/l of EPPC in the volume 15 ml/kg of animal body weight) allows to achieve the EPPC concentration of 16,000 cells per μl of blood, that is twice as many as the upper limit of a normal quantity of leukocytes in human blood and 80,000 times more than after administration of the cell dose that is applied in the clinical practice nowadays (Snigir N. V. Use of Hematopoietic Cells of Human Embryonic Liver for the Treatment of Cytostatic Myelodrepression in Oncological Patients: Manuscript. Dissertation . . . M.D./14.01.07-Oncology-Kyiv, 2001.-20 P.).

The control animals received an appropriate volume of 5% dimexide solution in Hanks medium. 376 white rats were used in the experiments.

Operative notes of skin allotransplantation in pairs. Two rats were simultaneously anesthetized (sodium pentobarbital−40 mg per kg of body weight). Hair was carefully removed from the back using scissors. Skin was prepared with 96% alcohol and alcohol iodine solution. According to a pattern (diameter−5 cm), the borders of the skin pieces were marked with concentrated iodine. Skin pieces were separated and, then, the skin allotransplantation was performed in pairs (one animal from the pair was from the control group, another was one that received EPPC) closing skin borders with interrupted single-row sutures.

Operative notes on spleen allotransplantation in pairs. Two rats were simultaneously anesthetized using sodium pentobarbital in an amount of 40 mg per kg of body weight. Hair was carefully removed from the abdomen using the scissors. The skin was treated with 96% alcohol and alcohol iodine solution. After midline laparotomy, ½ of spleen was removed from both rats, and encapsulated, and a pair spleen allotransplantation was performed into epiploon (one animal from the pair was from the control group, and another was one that received EPPC). Skin and muscles were closed by interrupted single-row sutures.

Isolation of cells. On the first, second, third, forth, fifth, sixth, seventh, tenth and fifteenth day after operation, under Nembutal narcosis, thymus, bone marrow, mesenteric lymph nodes and spleen were removed from animals. Organs were crushed and the cells were isolated using soft pipetting of tissues in 0.9% sodium chloride buffered with phosphates (NCBP) with a subsequent filtration of the received suspension through a capron filter (pore diameter is 200 μm).

Caspase-3 and caspase-8 activity in cell lysates isolated from thymus, bone marrow, spleen and lymph nodes was determined using BioVision (USA) reagents and recommendations with registration of indices using multi-scanner “Uniplan-M” (Russia). Concentration of protein in cell suspension was determined using Luori method. In order to determine a tissue.

EPPC localization, after their intravenous or intraperitoneal administration, the EPPC membrane staining was performed with a linkom fluorescent stain “PKH-67” according to the manufacturer's instructions (Sigma).

In order to study apoptosis, the cells were stained according to Shimizu S. et al. methodology (Shimizu S., Eguchi Y., Kamiike W. Involvement of ICE Family Proteases in Apoptosis Induced by Reoxygenation of Hypoxic Hepatocytes II Amer. J. Physiol.-1996.-Vol. 271.-P. G949-G958.). The solution of nuclear dyes “Hechst 33342” and propidium iodide (final concentration is 15 μM/l) were added to the cell suspension and incubated for 15 minutes at room temperature in darkness. For washing out the dyes, (8:1) (NCBP) solution was added to the cell suspension and centrifuged under 900 g for 7 min.

Specimen preparation. Stained cell suspension was placed on the glass and, after drying, fixed in formalin vapors for 10 min, washed out, dried and placed in Polymount.

Cell calculation. Specimens were studied using a luminescent microscope “ML-2” (immersion lens, 100 times magnification). The number of apoptotic cells and bodies was calculated, as well as the cells with an expressed condensation of nuclear chromatin of apoptotic type (at least, 400 cells in each specimen were studied for apoptotic properties). Microphotography. In the work, a color negative “Kodak Gold 100” film (ISO 100/21) was used with exposure, during photographing of the luminescence, from 30 to 120 sec, and of the phase-contrast image—from 4 to 15 sec.

The results were statistically processed using PC IBM Pentium III having installed “BioStat” software (Glants S. Medico-Biological Statistics.-M.: Practice, 1999.-459 P.).

### Results and Discussion

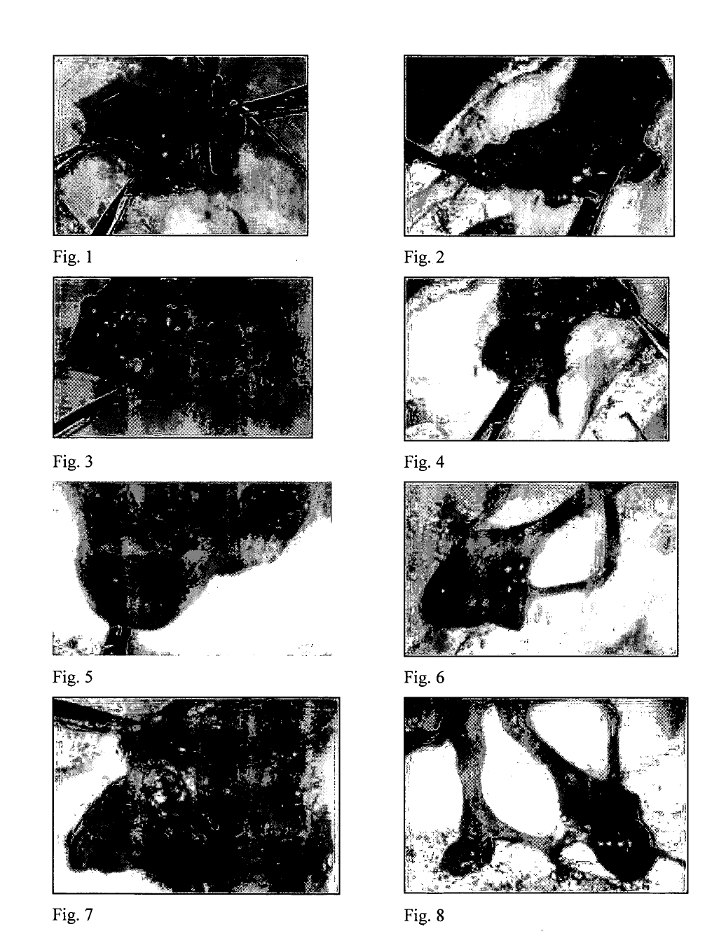

In the rats which received EPPC during the experiment, the skin allotransplants rejection was not observed, whereas, in the control group of control animals, the skin allotransplants rejected on the third (5% of animals), the seventh (75% of cases) and the ninth (20% of rats) observation day. Control laparotomy was performed in rats after 1 and 2 months of spleen allotransplantation and revealed the engraftment of the latter only in the animals, which received EPPC (FIG. 1-8). In order to exclude any possibility of EPPC-mediated immunodepressive effect and to confirm their influence to the development of immunological tolerance (e.g., a non-reactivity of the immune system to a specific antigen induced by the same antigen), a series of experiments was performed as shown in FIG. 9. At the stage A, experimental animals of the first group received EPPC (EPPC recipients), rats of the second group received Hanks solution (control animals). Under Nembutal narcosis, ½ spleen allotransplantation was performed in pairs (see operative notes). After 2 months, a laparotomy revealed the allospleen engraftment in the animals, which received EPPC, and spleen allotransplant rejection in the rats of the control group (FIG. 9, B, FIGS. 10-11). In the same pairs of rats, at the next stage of the experiment, the allotransplantations of the skin and a second half of the spleen were simultaneously performed (FIG. 9, C). After 2 weeks, the engraftment of the second half of allospleen in animals of the first group with an intensive allospleen rejection reaction in the control rats (FIGS. 14-15) was observed. It is important that such skin allotransplant rejection was observed in all animals of both groups (FIG. 9, D, FIGS. 14-15), i.e. the experimental results evidence that an intravenous EPPC administration does not result in an immunosuppression, but in the appearance of a stable immunological tolerance to that alloantigen, which was present in the organism at the time of EPPC administration.

It is known that an immunological tolerance to own antigens is not genetically determined, but established in the process of ontogenesis process according to mechanisms of positive and negative selection of T-lymphocytes in thymus. A necessary condition for induction of an immunological tolerance is not only the presence in thymocytes of a certain degree of receptors' affinity to the molecules of the main histocompatibility complex, but also a contact of maturing T-lymphocytes with peptide material of own tissues, expressed on epithelial, dendritic and interdigitate thymus cells (Roitt A., Brostoff J, Male. D. Immunology/Translation from English by V. I. Kondror, A. N. Mats, L. A. Pevnitskyy, M. A. Serova.-M.: Mir, 2000.-592 P.). Thymus “loading” with its own antigen material occurs in the process of embryogenesis, whereupon hematothymic barrier structures are formed (Khlystova Z. S. Formation of human fetus immunogenesis system/AMS USSR.-M.: Medicine, 1987.-256 P.). In humans, a hematothymic barrier is intrauterine, in rodents, it forms in 5 days after the birth. It should be noted that in rats, an immunological tolerance could be induced by the induction of a foreign antigen within the first five days of the neonatal period (Vershygora A. E. Basic immunology.-K.: High school, 1989.-736 P.; Anderssen C., Stocker E., Klinz F. J. et al. Nestin-Specific Green Fluorescent Protein Expression in Embryonic Stem Cell-Derived Neural Precursor Cells Used for Transplantation//Stem Cells.-2001.-Vol. 19.-P. 419-424.). Therefore, for implementation of initial stages of reinstallation of the control system of antigen homeostasis in an adult organism, it is necessary to integrate EPPC not only with bone marrow cells, but also with thymus cells, which are located beyond the barrier. It means that a pre-requisite is an increase of penetrability of the hematothymic barrier for an allogenic cell and peptide material.

Results of our experiments evidence that only in 1 hour after intravenous and in 1.5 hour after intraperitoneal administration of EPPC, membranes of which were stained with PKH 67, the marked cells are revealed among thymus cells (FIGS. 16-25). In addition, PKH 67-Positive cells have localized in lymph nodes, spleen and bone marrow. In the latter, their presence is proven by the similar experiments on other animals. (Askenasy N., Zorina T., Farkas D. L., Shalit I. Transplanted Hemotopoietic Cells Seed in Clusters in Recipient Bone Marrow in vivo 1/Stem Cells.-2002.-Vol. 20. -P. 301-310.). It should be noted, that PKH 67 is a linkorn dye, which does not change natural properties of the cell membrane (Askenasy N., Farkas D. L. Antigen Barriers or Available Space do not Restrict in situ Adhesion of Hemopoietic Cells to Bone Marrow Stroma II Stem Cells.-2002.-Vol. 20.-P. 301-310.). The importance of this fact is that the presence of EPPC in thymus only in 1 hour after intravenous administration proves their ability to increase the penetrability of the hematothymic barrier. Further EPPC differentiation, as it was established by other authors (Takatsugu Y., Masahide Y., Seiji K., Yoko K., Yoshiyuki N., Shigeaki I., Yukio T. In vitro Differentiation of Embryonic Stem Cells into Hepatocyte-Like Cells Identified by Cellular Uptake of Indocyanine Green II Stem Cells.-2002.-Vol. 20.-P. 146-154.) is determined purely by their microehvironment, first of all, stromal (Bianco P., Riminucci M., Gronthos S., Robey P. G. Bone Marrow Stromal Stem Sells: Nature, Biology and Potential Applications II Stem Cells.-2001.-Vol. 19.-P. 180-192.). EPPC differentiation in thymus into epithelial, interdigitate and dendritic cells on the background of alloantigen load leads to expression of alloantigens (in our experiments—skin and spleen), which join in the process of negative selection of T-lymphocytes. Simultaneously, in the de novo formed cells of the thymus, MHC class I and II molecules that are genetically determined in EPPC are expressed. The situation is similar to the immunological tolerance of tetraploid hermaphrodites to the antigens of both parents—in the organism of the EPPC recipient, a double standard of histocompatibility molecules is established. We should note that it is shown it various experimental lines of animals that tetraparental allophonic mouse chimeras have histocompatibility antigens from both parental lines and do not reject their transplants (Vershygora A. E. Basic Immunology.-K.: Higher School Publishers, 1989.-736 P.).

At the same time, EPPC in bone marrow under the influence of stromal environment, differentiate into hematopoietic progenitor cells, including lymphopoesis progenitors. Under such conditions, a crucial question for reinstallation of the control system of antigen homeostasis of the organism during EPPC administration is the conflict of two co-existing mature immune competent effector cells in the organism. The absence of local and system manifestations of immune inflammation in the presence of short-term positive clinical effect after administration of hematopoietic stem cells of human embryo liver in small doses (Novitskaya A. V. The Treatment of Patients Suffering from Diabetes Mellitus with Immune and Hematological Disorders of the Human Embryo Liver by Hematopoietic Cells: Abstract. Dissertation . . . candidate of medicine/14.01.14-endocrinology-Kyiv, 2000.-20 P.) indicates that the conflict between two different groups of mature lymphocytes may result in apoptosis. It is known that blood-forming progenitors are always ready to develop apoptosis and require a protective action of cytokines for protection from a programmed death. Therefore, in the blood system pathology, an apoptosis plays a significant role, in particular, a pathogenesis of many cytopenias and pancytopenias is connected with its increase (Vladimirsky E. B. Mechanisms of Apoptic Cell Death II Hematology and Transfusion.-2002.-V. 47, No 2. -P. 35-40.).

According to the results of our experiments in the specimens of thymus, bone marrow, spleen and lymph nodes tissues, after skin and spleen allotransplantation in pairs cells have been observed in all animals being at different stages of death according to apoptosis mechanisms (FIGS. 26-32). Study of changes in dynamics in relation to a number of apoptotic cells in central and peripheral organs of the rats' immune system proved that, in thymus of the animals, which received EPPC, an apoptosis intensity significantly exceeded the control indices on the first, third, sixth and fifteenth observation days (FIG. 30) with the presence of an exponential trend for approximation of the results, whereas the apoptosis dynamics in thymus of the control rats was characterized by a logarithmic trend (FIG. 33). The number of apoptotic cells in bone marrow also dramatically increased in the animals, which received EPPC demonstrating a linear trend of growing dynamics, in contrast to the control, where approximation of dynamics of changes in the number of apoptotic cells revealed an exponential trend with a direct decrease vector (FIG. 34). Simultaneously, in thymus and in bone marrow, the activity of caspase-8 and caspase-3 increased (FIGS. 26-41).

The role of apoptosis in the determination of cell population structure is especially important for the immune system cells. The basis of selection of thymocyte clones is the ability of their membrane receptors to recognize ligands on the surface of thymus stroma cells. In young cells of CD4⁺CD8⁺ phenotype, due to a weakness of reparation system, multiple DNA ruptures are accumulated. This leads to the death program realization, if the cells do not receive a signal from stroma epithelial cells, which causes an increase of expression of Bc 1-2 protective factor. Such signals are available only for those clones of thymocytes, which are able to recognize autological molecules of histocompatibility (positive selection). On the other hand, cells receive a signal for apoptosis, when they recognize autological peptides, built in autological molecules of histocompatibility (negative selection). As a result, the structure of the initial thymocytes population, which have a great variety of antigen recognizing receptors, including potentially dangerous ones, is corrected and mature T-lymphocytes respond only to foreign peptides, which are present with autological molecules of histocompatibility (Roitt A., Brostoff J., Male D. Immunology./Translation from English by V. I. Kandror, A. N. Mats, L. A. Pevnitskyy, M. A. Serova.-M.: Mir, 2000.-592 P.).

Thus, in case of EPPC integration into thymus and bone marrow and their differentiation into specialized cells, a type of which is determined by a stromal environment, none of the above mechanisms of T-lymphocyte maturation in the process of natural immunological tolerance induction is affected, and inclusion of antigens of transplanted organs into the de novo antigen presenting thymus cells expands the spectrum of “own” antigens that provide for a specific non-reactivity to allotransplants.

To answer the question, whether the replacement of immunocompetent cells by the new ones really occurs, or it is only a renewal of the control system for antigen homeostasis in the organism as a consequence of MHC (HLA) double standard formation, it should have been addressed how EPPC behave with different haplotypes under the conditions of co-existence in one cell suspension. The study of apoptosis dynamics revealed its dramatic intensification during 14 hour-long incubation of EPPC suspension mixed with different haplotypes (EPPC isolated from different embryos) compared with indices obtained during an isolated incubation of homogeneous EPPC suspensions (FIGS. 35-41). This finding proves that EPPC have systems for recognition of such cells with a different haplotype and are able to realize the cell death program that is the basis for replacement of the stem pool of bone marrow cells into a new one, subject to administration of EPPC in megadoses into the organism.

### CONCLUSIONS

Results of these biological experiments prove the finding of a new phenomenon in biology and medicine, that is the induction of immunological tolerance with megadoses of embryonic pluripotent progenitor cells (EPPC) by means of formation of a new basis of immunocompetent cells with simultaneous de novo installation of the system of control of the major histocompatibilty complex (MHC) (Kukharchuk-Radchenko-Sirman effect).

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All references cited herein, including all patents, published patent applications, and published scientific articles, are incorporated by reference in their entireties for all purposes.

1. A method of reinstallation of the control system for antigenic homeostasis of a mammalian organism, characterized in that, instead of the antigen selection of donor tissues and immune suppression, the immunological tolerance of the recipient to allotransplants is achieved by the administration of embryonic pluripotent progenitor cells (EPPC) which form a new basis of immunocompetent cells controlling the antigenic homeostasis of the organism.
